(19) United States
(12) Patent Application Publication
(10) Pub. No.: US 2018/0113128 A1
(43) Pub. Date: Apr. 26, 2018

(54) DETECTION OF URINARY TRACT INFECTIONS

(71) Applicant: ISI Life Sciences, Inc., Newport Beach, CA (US)

(72) Inventors: Gerald F. SWISS, San Diego, CA (US); Robert M. MORIARTY, Michiana Shores, IN (US); Richard PARIZA, Chicago, IL (US); David WHITE, Newport Beach, CA (US)

(73) Assignee: ISI Life Sciences, Inc., Newport Beach, CA (US)

(21) Appl. No.: 15/789,825

(22) Filed: Oct. 20, 2017

Related U.S. Application Data
(60) Provisional application No. 62/410,809, filed on Oct. 20, 2016.
(60) Provisional application No. 62/415,396, filed on Oct. 31, 2016.

(51) Int. Cl. G01N 33/569; C12Q 1/04

(52) U.S. Cl. CPC G01N 33/56911; C12Q 1/04; A61K 9/0034; B01J 2219/00648; B01J 2219/00576

(57) ABSTRACT

Disclosed are compositions, methods, and kits that are directed to detection of urease expressing bacterial urinary tract infections and, in particular, those associated with the use of urinary catheters.

### CROSS-REFERENCE TO RELATED APPLICATIONS

This application claims priority to U.S. Provisional Application No. 62/410,809, filed Oct. 20, 2016; and U.S. Provisional Application No. 62/415,396, filed Oct. 31, 2016, the contents of each of which is incorporated herein in its entirety by reference.

### FIELD OF THE DISCLOSURE

This invention is directed, in part, to detection of urinary tract infections and, in particular, those associated with the use of urinary catheters. In particular, this technology provides for detection of bacterial infections by using a label capable of producing a distinct detectible signal indicative of a urinary tract infection.

### STATE OF THE ART

Urinary tract infections (UTIs) are common infections generally in women, but also common in men and women fitted with a urinary catheter, the latter of which are referred to as catheter associated urinary tract infections (CAUTIs). Hospital acquired UTIs are especially troublesome, as many bacteria are now or suspected of soon to be antibiotic resistant. As to hospital acquired UTIs, it is recognized that from about 15 to 25 percent of admitted patients equipped with a urinary catheter will develop a UTI. Moreover, the CDC reports that approximately 75% of all hospital acquired infections are UTIs.

In addition to hospital acquired UTIs, it is recognized that the length of time a urinary catheter remains in the body is directly related to the likelihood of developing a CAUTI. For example, estimates provide that from 10 to 50% of patients undergoing short-term catheterization (up to 7 days) and nearly 100% of patients undergoing long-term catheterization (>28 days) will develop a CAUTI. Such long term use of urinary catheters is a problem not limited to hospitals but also a problem in nursing homes, extended care facilities, and at-home patients. Such uses outside of the hospital setting are aggravated by the fact that the level of care in these other settings is not likely to be as stringent as in a hospital setting.

Bacteria that generate UTIs can be ascribed to those that produce an enzyme such as a urease that generates ammonia in the patient's urine. Such offending bacteria are represented by Proteus spp. and especially Proteus mirabilis. Other bacteria are that can generate UTIs include E. coli, Enterococcus spp. and the like. As a urinary tract infection develops, esterases are released into the urine as part of the inflammatory sequale. Such esterase release is symptomatic of any bacterial based UTI.

The diagnosis of a UTI by current clinical criteria alone has a reported error rate of about 33%. See, Deutsches Arzteblatt International, 107(21):361-367 (2010). Such an error rate is unacceptable and often leads to more advanced UTIs with a corresponding increase in mortality. Annually, it is estimated that 13,000 individuals die in the US from unresolved UTIs, many of which have progressed to sepsis.

Currently, rapid UTI test kits are available such as nitrite dipsticks. However, these dipsticks have been evaluated to be unreliable and not suitable for use for patient evaluation. Mambatta et al., J Family Med Prim Care. 2015 Apr-Jun; 4(2): 265-268 (available at www.ncbi.nlm.nih.gov/pmc/articles/PMC4408713/).

Accordingly, there is a serious and unmet need to rapidly assess the presence of a urinary tract infection in patients and especially those equipped with a urinary catheter.

### SUMMARY OF THE DISCLOSURE

This invention provides for a facile, non-invasive assay for assessing the presence of a UTI in a patient merely by measuring for the generation of a signal that correlates the urine of a patient to the presence of a UTI. In one embodiment, the label is a pro-label that contains an ester bond that is retained as an ester bond in the patient's urine in the absence of UTI. However, in the presence of urine from a patient with a UTI, the ester bond of the pro-label is cleaved to convert the pro-label a label capable of generating a distinct signal. That distinct signal allows for rapid and accurate indicia of the presence of a UTI in that patient.

In one embodiment, this invention provides for a method for detecting a urinary tract infection (UTI) in a patient suspected of being afflicted with a UTI which method comprises:
contacting urine of said patient with a pro-label having one or more ester functionalities wherein said ester functionalities remain intact in the presence of normal urine but are cleaved in the presence of urine associated with a UTI to provide a label that is capable of providing for a distinct detectible signal evidencing the presence of a UTI;
maintaining said pro-label in contact with said urine under conditions wherein the ester bond or bonds is/are cleaved in the presence of a UTI;
assessing for the presence or absence of said signal; and
correlating the presence or absence of said signal to the presence or absence of a UTI in said patient.

In one embodiment, this invention provides for a method for detecting a urinary tract infection (UTI) in a patient suspected of being afflicted with a UTI which method comprises:
contacting urine of said patient with a pro-fluorescent label having one or more ester functionalities wherein said ester functionalities remain intact in the presence of normal urine but are cleaved in the presence of urine associated with a UTI to provide a label that is capable of generating a distinct detectible fluorescent signal evidencing the presence of a UTI;
maintaining said pro-fluorescent label in contact with said urine under conditions wherein the ester bond or bonds is/are cleaved in the presence of a UTI;
assessing for the presence or absence of said fluorescent signal; and
correlating the presence or absence of said signal to the presence or absence of a UTI in said patient.

In one embodiment, this invention provides for a method for detecting a urinary tract infection (UTI) in a patient suspected of being afflicted with a UTI which method comprises:
contacting urine of said patient with a pro-fluorescent fluorescein label having one or more ester functionalities wherein said ester functionalities remain intact in the presence of normal urine and are cleaved in the presence of urine associated with a UTI to provide a fluorescent fluorescein label capable of providing a distinct detectible signal evidencing the presence of a UTI;
maintaining said pro-fluorescent fluorescein label in contact with said urine under conditions wherein the ester bond or bonds is/are cleaved in the presence of a UTI;
assessing for the presence or absence of said signal; and
correlating the presence or absence of said signal to the presence or absence of a UTI in said patient.

Other labels that can be used include pH indicators, isotope release labels, solid particles that affect light scattering, and the like provided that such labels can be covalently attached to an ester functionality.

In one embodiment, the label is a color indicator such as a dye or pH indicator that provides an intense non-natural color to urine. For example, the color indicator could provide for a red, brown, purple, blue, green or other color that is not naturally associated with urine. The label can be attached to solid particles such as beads via an ester bond. When contacted with urine from a patient with a UTI, the ester bond is cleaved and the color indicator is released from the solid support bound thereby altering the urine to a distinct unnatural color that provides clear visual evidence that the patient is afflicted with a UTI. Alternatively, the previously colored supports will lose their color upon cleaving of such ester bond(s) and such loss of color can be another aspect to evaluate for the presence or absence of a UTI. Other labels could be similarly attached.

Each of these methods employs compositions that contain a fissile (cleavable) ester bond that when bound to a label converts that label to a pro-label, wherein the fissile bond is broken by a component only found in urine from a patient suffering from a UTI. For example, the component can be ammonia/ammonium hydroxide that is generated by a urease-expressing bacteria. Alternatively or in combination, the component can be an esterase that is generated by the inflammatory sequale arising from a UTI. It is contemplated that such esterases are derived by lysing invading leukocytes thereby releasing leukocyte esterases which, in turn, are capable of cleaving ester bonds in the pro-labels used herein. See, e.g., www.rnceus.com/ua/uanile.html

In either case, the presence of ammonia or the esterase in the urine will rapidly break ester bonds in a manner that allows for conversion of the pro-label to the label into the urine. In turn, detection of the label evidences the presence of a UTI in that patient. Alternatively, the loss of labels from, for example, a support is also indicia of the presence of a UTI.

The methods of this invention are useful in detecting a UTI in a patient suspected of suffering from a UTI. In one preferred embodiment, the UTI is a CAUTI.

In one of its composition aspects, this invention provides for a label comprising a plurality of ester bonds that convert the label into a pro-label and maintaining said composition in urinary catheter or a urine collection bag.

In one embodiment, the ester bonds on said pro-label are masked to prevent the label from generating a signal. In such an embodiment, pro-label remains unable to generate a signal unless the urine in contact therewith is obtained from a patient with a UTI that cleaves the ester bond(s).

In another embodiment, the pro-label retains its detectible signal but that label is bound to a support via an ester bond. The support retains such pro-labels in a first position within, for example, the catheter or urine bag (or other collection container). When in contact with normal urine, these pro-labels are retained on the support in said first position. When in contact with urine from a patient with a UTI, the ester bonds are cleaved releasing the label from the support and said first position and into the urine (said second position) thereby providing a detectible signal in the urine.

In either case, cleaving of the ester bond(s) on the pro-label serves to provide a clear indicia that components generated by a UTI are present in the urine.

In one embodiment, the detectable label is a fluorescent label. Preferred fluorescent labels are those that exhibit intense fluorescence at low concentrations. Suitable fluorescent labels include fluorescein, fluorescein derivatives, MR121, Alexa 594, Alexa 610, Alexa 680, Alexa 690, Alexa 700 and like including the compound of the formula (available from Sigma Aldrich, St. Louis, Mo., USA):

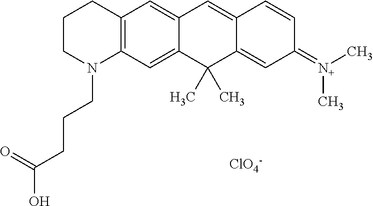

which has excitation wavelength is 610 nm and the emission wavelength is 635 nm.

Also, phycoerythrobilin is a well-known fluorophore of the structure

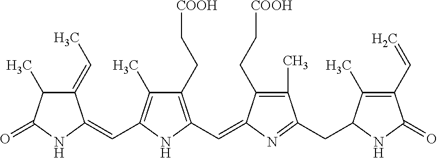 [SMILES: C=CC1=C(C)C(CC2=N/C(=C/c3[nH]c(/C=C4/NC(=O)C(C)/C4=C\C)c(C)c3CCC(=O)O)C(CCC(=O)O)=C2C)NC1=O]

where the excitation wavelength is 576 nm and the emission wavelength is 586 nm.

Indeed, any label that can be bound to, e.g., a support via an ester bond can be used. Further, any label having either a hydroxyl or carboxyl group can be used and coupled to a solid support having a complementary carboxyl or hydroxyl group. Formation of ester or carbonate groups is well known in the art.

The support is any material that, in addition to forming an ester bond, can be retained in a chamber, that is to say a chamber defined by an enclosed structure that allows for passage of fluids there through while retaining the solid within said chamber. For example, chambers that contain a pore size of say 10, 50 or 100 microns can be used provided that the solid particles are larger than 10, 50 or 100 microns respectively. Such a chamber allows for flow of urine there through while retaining the solid particles within the chamber.

As before, the solid particles contain a reactive functionality that allows for ester bond formation between the label and the solid support. Examples of hydroxyl containing supports include poly (2-hydroxyethylmethacrylate), cellulose, other polysaccharides, partially hydrolyzed polyvinyl acetate, hydrogels including polyglycerol hydrogels, and the like.

Examples of carboxyl containing supports include, by way of example only, carboxymethylcellulose, polyacrylic acid, polymethacrylic acid, carboxymethylated chitin, and the like.

In another embodiment, labels attached to a support via ester groups are placed into a chamber that is integrated into either a catheter or a urine collection bags or a connector therebetween. In one embodiment, the solid support is sized in a manner so as to allow fluid passages there through while providing for intimate contact of the fluid with the solid supports in the chamber. In one particular embodiment, the chamber is placed in a connector between the catheter and the urine collection bag wherein the chamber is fitted with a proximal frit and a distal frit to entrap the solid supports within the frits and walls of the connector of the chamber while allowing fluid passage through the chamber. In the presence of urine from a patient with a UTI, the ester bonds will cleave thereby releasing the label into the fluid flow and into the urine collection bag. As such, the accumulation of label in the urine collection bag allows the clinician to evaluate the presence of the UTI without any handling of bodily fluids.

In the case of a urease generating bacterial infection, it is contemplated that the signal generated by the UTI can be amplified by including urea particles such as microparticles in the chamber. Such microparticles, in the presence of any urease, will generate additional ammonia in the urine. The resulting ammonia will enhance the early detection of a urinary tract infection that expresses urease. The amplification of signal also allows for the detection of incipient urinary tract infection. The presence of urease is direct evidence of a UTI whereas the presence of ammonia is indirect evidence that provides a strong correlation to the presence of a UTI.

In another of its aspects, there is provided a chamber comprising an inner wall and an outer wall, wherein the inner wall defines a lumen and the outer wall of said chamber fits within, e.g., a catheter, a connector between the catheter and the urine collection bag or can be inserted into a urine collection bag. At least a portion of the chamber is comprises permeable membranes preferably proximally and distally spaced within the lumen so as to define a flow through port in the chamber. The interior lumen of the chamber contains solid particles wherein said particles comprise labels reversibly bound to the particles via ester bonds that are broken by a component distinctly found in urine from a patient suffering from a UTI such that said component cleaves the bond thereby releasing the label into the urine flow.

In one embodiment, the chamber further comprises urea particles such as microparticles comprising urea.

In one embodiment, the chamber is defined as part of a connector that links the terminal end of the catheter to the inlet port of the urine collection. Preferably, inside the connector are the proximal and distal frits or membranes that define the chamber.

In another embodiment, the chamber can be positioned within the lumen of the catheter and preferably at the distal end of the catheter that links the catheter to the collection bag. Again, the chamber preferably contains proximal and distal frits.

In another embodiment, the chamber can be positioned as part of the collection bag such as the inlet port into the bag. Again, the chamber contains proximal and distal frits.

In another embodiment, the pro-label whether attached to a solid support or not can be added directly to the urine bag.

The solid support can be in any form that permits contact with the fluid passing through the chamber. In one embodiment, the solid support is sized and shaped so as to provide a packing density that creates voids within the chamber that allow passage of urine through the support while intimately contacting the support. The particular size and shape is not critical and can be in the form of small spheres, irregularly shaped particles, a porous membrane, a vertical fan, and the like. The solid support can be intermixed with other material that enhances water permeability through the chamber. Such material includes inert components such as sand, inert polymeric particles, glass beads, and the like. The packing density can be adjusted as necessary by use of such inert materials as such is well known in the art.

In another embodiment, the chamber can take any form suitable for use as described herein including a cartridge, a cassette, a canister or a porous plug.

In another embodiment, there is provided a method for detecting the presence of urinary tract infection. The method comprises contacting a urine sample suspected of such a UTI with a pro-label either attached to a solid support that contains labels bound thereto through a fissile bond or pro-label compounds placed into a urine collection bag wherein the fissile bond is only broken by contact with urine from a patient suffering from a UTI; and assessing the presence of a UTI based on the presence or absence of signal generated by label.

### DESCRIPTION OF THE DRAWINGS

The patent or application file contains at least one drawing executed in color. Copies of this patent or patent application publication with color drawing(s) will be provided by the Office upon request and payment of necessary fee.

FIG. 1 illustrates the lack of fluorescein fluorescence in a urine sample containing fluorescein dioctanoate but no urease.

FIG. 2 illustrates the presence of fluorescein fluorescence in a urine sample containing fluorescein dioctanoate and urease.

FIG. 3 illustrates the absence and presence of fluorescein fluorescence in a urine samples containing fluorescein diesters incubated with either P. mirabilis or E. Coli.

FIGS. 4A and 4B illustrate that fluorescein dioctanoate will convert to fluorescein and fluoresce when incubated with blood lysate whereas control does not.

### DETAILED DESCRIPTION OF THE DISCLOSURE

The invention described herein is directed to compositions, methods and kits for detecting urinary tract infections in patients and, in particular, in patients equipped with a urinary catheter.

### Definitions

As used herein, the following definitions shall apply unless otherwise indicated. Further, if any term or symbol used herein is not defined as set forth below, it shall have its ordinary meaning in the art.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of this invention. Procedures for inserting such labels into the compounds of this invention will be readily apparent to those skilled in the art based on the disclosure herein.

The term “UTI” refers to a urinary tract infection. The term “CAUTI” refers to a catheter associated urinary tract infection.

The term “label” refers to any material that produces or can be modified to produce a distinct signal that is detectible and correlates to the presence of a urinary tract infection. Suitable signals include by way of example only fluorophores, chromophores, light scattering particles, enzymes that produce detectible signals such chemiluminescence, bioluminescence, fluorescence, etc., as well as co-factors of enzymes, etc.

The term “chromophore” refers to any compound that produces a detectable signal including pH indicators, fluorophores, chemiluminescence, bioluminescence, and the like.

The term “fluorophore” refers to any compound that fluoresces in the presence of light and preferably UV light.

The term “carboxyl-containing label” refers to labels that contain one or more carboxyl groups.

The term “hydroxyl-containing label” refers to labels that contain one or more hydroxyl groups.

The term “linker” refers to a group that links one portion of a molecule to another portion of a molecule. Such linkers are typically from 1 to 30 atoms which comprise carbon, nitrogen, oxygen, sulfur, —S(O)- and —S(O)₂- wherein the carbon and nitrogen atoms can have hydrogen, carbon, —S(O)- and —S(O)₂- and other groups attached thereto.

The term “urease” refers to any enzyme that is capable of converting urea to ammonia.

The term “bacteria that express urease” refers to any bacteria that is capable of expressing urease including Proteus ssp. and any other bacteria.

The term “solid support” refers to a material that is preferably inert and which is capable of binding labels thereto through a fissile bond. Such materials include those containing carboxyl groups as well as hydroxyl groups.

The term “alkyl” refers to monovalent saturated aliphatic hydrocarbyl groups having from 1 to 20 carbon atoms and preferably 1 to 10 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

The term “substituted alkyl” refers to an alkyl group having from 1 to 5, preferably 1 to 3, or more preferably 1 to 2 substituents selected from the group consisting of alkoxy, substituted alkoxy, acyl, acylamino, amino, substituted amino, aryl, substituted aryl, aryloxy, substituted aryloxy, carboxyl, carboxyl ester, cyano, cycloalkyl, substituted cycloalkyl, cycloalkyloxy, substituted cycloalkyloxy, halo, hydroxy, heteroaryl, substituted heteroaryl, heteroaryloxy, substituted heteroaryloxy, heterocyclic, substituted heterocyclic, heterocyclyloxy, substituted heterocyclyloxy, and nitro, wherein said substituents are defined herein.

The term “alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

The term “substituted alkoxy” refers to the group —O-(substituted alkyl).

The term “acyl” refers to the groups alkyl-C(O)—, substituted alkyl-C(O)—, cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—.

The term “acyloxy” refers to the group —O-acyl.

The term “acylamino” refers to the group -acyl-amino.

The term “amino” refers to the group —NH₂.

The term “substituted amino” refers to the group —NR³¹R³² where R³¹ and R³² are independently selected from the group consisting of hydrogen, alkyl, substituted alkyl, aryl, substituted aryl, cycloalkyl, substituted cycloalkyl, heteroaryl, substituted heteroaryl, heterocyclic, substituted heterocyclic, and wherein R³¹ and R³² are optionally joined, together with the nitrogen bound thereto to form a heterocyclic or substituted heterocyclic group, provided that R³¹ and R³² are both not hydrogen.

The term “aryl” or “Ar” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

The term “substituted aryl” refers to aryl groups which are substituted with 1 to 5, preferably 1 to 3, or more preferably 1 to 2 substituents selected from the group consisting of alkyl, substituted alkyl, alkoxy, substituted alkoxy, acyl, acylamino, amino, substituted amino, aryl, substituted aryl, aryloxy, substituted aryloxy, carboxyl, carboxyl ester, cyano, cycloalkyl, substituted cycloalkyl, cycloalkyloxy, substituted cycloalkyloxy, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, and nitro.

The term “aryloxy” refers to the group —O-aryl.

The term “substituted aryloxy” refers to the group —O-(substituted aryl).

The term “carbonyl” refers to the divalent group —C(O)- which is equivalent to —C(=O)—.

The term “carboxy” or “carboxyl” refers to —COOH or salts thereof.

The term “carboxyl ester” or “carboxy ester” refers to the groups —C(O)O-alkyl, —C(O)O-substituted alkyl, —C(O)O-aryl, —C(O)O-substituted aryl, —C(O)O-cycloalkyl, —C(O)O-substituted cycloalkyl, —C(O)O-heteroaryloxy, —C(O)O-substituted heteroaryl, -non-heterocyclic, and —C(O)O-substituted.

The term “cyano” refers to the group -C≡N.

The term “cycloalkyl” refers to a saturated or unsaturated but nonaromatic cyclic alkyl groups of from 3 to 10 carbon atoms having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more rings can be aryl, heteroaryl, or heterocyclic provided that the point of attachment is through the non-aromatic, non-heterocyclic ring saturated carbocyclic ring.

The term “substituted cycloalkyl” refers to a cycloalkyl group having from 1 to 5 or preferably 1 to 3 substituents selected from the group consisting of oxo, alkyl, substituted alkyl, alkoxy, substituted alkoxy, acyl, acylamino, acyloxy, amino, substituted amino, aryl, substituted aryl, aryloxy, substituted aryloxy, carboxyl, carboxyl ester, cyano, cycloalkyl, substituted cycloalkyl, cycloalkyloxy, substituted cycloalkyloxy, halo, hydroxy, heteroaryl, substituted heteroaryl, heteroaryloxy, substituted heteroaryloxy, heterocyclic, substituted heterocyclic, heterocyclyloxy, substituted heterocyclyloxy, and nitro.

The term “cycloalkyloxy” refers to —O-cycloalkyl.

The term “substituted cycloalkyloxy” refers to —O-(substituted cycloalkyl).

The term “halo” or “halogen” refers to fluoro, chloro, bromo and iodo and preferably is fluoro or chloro.

The term “hydroxy” or “hydroxyl” refers to the group —OH.

The term “heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, indolyl, thiophenyl, and furanyl. In some embodiments, preferred heteroaryls include 5- or 6-membered non-oxygen containing heteroaryls. In some embodiments, preferred heteroaryls include 5- or 6-membered heteroaryls that exclude oxazoles. In some embodiments, a nitrogen-containing heteroaryl group is attached through a nitrogen on the heteroaryl group.

The term “substituted heteroaryl” refers to heteroaryl groups that are substituted with from 1 to 5, preferably 1 to 3, or more preferably 1 to 2 substituents selected from the group consisting of the same group of substituents defined for substituted aryl.

The term “heteroaryloxy” refers to —O-heteroaryl and “substituted heteroaryloxy” refers to —O-substituted heteroaryl.

The term “heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

The term “substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

Examples of heterocycle and heteroaryl include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl, 1,1-dioxothiomorpholinyl, piperidinyl, pyrrolidine, and tetrahydrofuranyl.

The term “heterocyclyloxy” or “heterocycloalkyloxy” refer to the group —O-(heterocyclyl).

The term “substituted heterocyclyloxy” or “substituted heterocycloalkyloxy” refer to the group —O-(substituted heterocyclyloxy.

The term “heterocyclylthio” or “heterocycloalkylthio” refer to the group —S-(heterocyclyl).

The term “substituted heterocyclylthio” or “substituted heterocycloalkylthio” refer to the group —S-(substituted heterocyclyl).

The term “heteroarylthio” refers to the group —S-(heteroaryl).

The term “substituted heteroarylthio” refers to the group —S-(substituted heteroaryl).

The term “nitro” refers to the group —NO₂.

The term “oxo” refers to the atom (═O) or (—O⁻).

The term “spiro ring systems” refers to bicyclic ring systems that have a single ring carbon atom common to both rings.

Stereoisomers of compounds (also known as optical isomers) include all chiral, dl, stereoisomeric, and racemic forms of a structure, unless the specific stereochemistry is expressly indicated. Thus, compounds used in this invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of this invention.

The compounds of this invention may exist as solvates, especially hydrates. Hydrates may form during manufacture of the compounds or compositions comprising the compounds, or hydrates may form over time due to the hygroscopic nature of the compounds. Compounds of this invention may exist as organic solvates as well, including DMF, ether, and alcohol solvates among others. The identification and preparation of any particular solvate is within the skill of the ordinary artisan of synthetic organic or medicinal chemistry.

The term “tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

Unless indicated otherwise, the nomenclature of substituents that are not explicitly defined herein are arrived at by naming the terminal portion of the functionality followed by the adjacent functionality toward the point of attachment. For example, the substituent “alkoxycarbonylalkyl” refers to the group (alkoxy)-C(O)-(alkyl)-.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

It is understood that the above definitions are not intended to include impermissible substitution patterns (e.g., methyl substituted with 5 fluoro groups). Such impermissible substitution patterns are well known to the skilled artisan.

### Methods

The methods of this invention are predicated on the discovery that ester bonds are stable in normal urine and pro-labels that contain an ester bond will remain intact for prolonged periods of time in the presence of such urine. This invention is further predicated on the discovery that ester bonds are cleaved in the presence of urine from a patient with a UTI, thereby converting the pro-label to a label capable of producing a distinct, detectible signal. Such cleaving of these bonds is due either to the generation of ammonia/ammonium hydroxide by a urease expressing bacteria or by the generation of esterases in the urine of patients with other bacterial based UTIs. The methods of this invention provide a facile assay for detecting a UTI in a patient merely by correlating the presence or absence of a label.

Accordingly, one such method is a method for detecting a urinary tract infection (UTI) in a patient suspected of being afflicted with a UTI which method comprises:
contacting urine of said patient with a pro-label having one or more ester functionalities wherein said ester functionalities remain intact in the presence of normal urine but are cleaved in the presence of urine associated with a UTI to provide a label that is capable of providing for a distinct detectible signal evidencing the presence of a UTI;
maintaining said pro-label in contact with said urine under conditions wherein the ester bond or bonds is/are cleaved in the presence of a UTI;
assessing for the presence or absence of said signal; and
correlating the presence or absence of said signal to the presence or absence of a UTI in said patient.

Another such method provides a method for detecting a urinary tract infection (UTI) in a patient suspected of being afflicted with a UTI which method comprises:
contacting urine of said patient with a pro-fluorescent label having one or more ester functionalities wherein said ester functionalities remain intact in the presence of normal urine but are cleaved in the presence of urine associated with a UTI to provide a label that is capable of generating a distinct detectible fluorescent signal evidencing the presence of a UTI;
maintaining said pro-fluorescent label in contact with said urine under conditions wherein the ester bond or bonds is/are cleaved in the presence of a UTI;
assessing for the presence or absence of said fluorescent signal; and
correlating the presence or absence of said signal to the presence or absence of a UTI in said patient.

Still another such method provides for a method for detecting a urinary tract infection (UTI) in a patient suspected of being afflicted with a UTI which method comprises:
contacting urine of said patient with a pro-fluorescent fluorescein label having one or more ester functionalities wherein said ester functionalities remain intact in the presence of normal urine and are cleaved in the presence of urine associated with a UTI to provide a fluorescent fluorescein label capable of providing a distinct detectible signal evidencing the presence of a UTI;
maintaining said pro-fluorescent fluorescein label in contact with said urine under conditions wherein the ester bond or bonds is/are cleaved in the presence of a UTI;
assessing for the presence or absence of said signal; and
correlating the presence or absence of said signal to the presence or absence of a UTI in said patient.

Each of these methods involves allowing the urine of a patient suspected of suffering from a UTI (including a CAUTI) to contact a pro-label having one or more ester bonds under conditions wherein the ester bonds remain intact in the absence of a UTI and are broken or cleaved in the presence of a UTI. Cleaving the ester bond converts the pro-label to a label that creates a distinct detectible signal indicating the patient is suffering from a UTI.

Without being limited to any theory, the invention is theorized to operate when bacteria in the urinary tract release an esterase and/or a urease. In the case of esterase release, these enzymes interact with ester bonds used herein and break or cleave these bonds. In the case of a urease, these enzymes interact with urea found in the patient's urine and convert the urea to ammonia and carbon dioxide. The resulting ammonia is nucleophilic and displaces/cleaves the ester bond. In both cases, the result of such enzymes is the conversion of the pro-label to a label.

In practicing the claimed methods, one merely needs to contact a sample of urine with a pro-label or a composition comprising the pro-label. The resulting sample is maintained for a sufficient period of time to allow for the ester bonds to be cleaved while maintaining the sample under ambient conditions such as those found in a urine collection bag. Typically, a suitable period of time is from less than a minute to about 30 minutes, and preferably from about 1 to 15 minutes. Afterwards, the clinician merely evaluates the urine for the presence of the label. If the sample is negative (no UTI), the urine will not evidence any signal generated by the label. If the sample is positive (a UTI), the urine will evidence a distinct detectible signal. As is apparent, a significant benefit of this invention is the ability to reliably test for the presence of a UTI without the need to contact the urine of the patient.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, N.Y., 1999, and references cited therein.

If the compounds of this invention contain one or more chiral centers, such compounds can be prepared or isolated as pure stereoisomers, i.e., as individual enantiomers or d(l) stereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as SigmaAldrich (St. Louis, Mo., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

Certain compounds used herein are commercially available such as fluorescein dioctanoate (AAT Bioquest, Sunnyvale, Calif., USA), fluorescein dibutyrate (Abcam, Cambridge, Mass., USA). Other esters can be prepared from known reagents using standard techniques whereby a label is acylated (esterified). For example, a hydroxyl containing labels can be acylated by reaction with a carboxyl containing acylating group such as a R—COOH where R is, by way of example, C₁-C₂₀ alkyl, C₂-C₂₀ alkenyl, C₂-C₂₀ alkynyl, C₁-C₂₀ substituted alkyl, C₂-C₂₀ substituted alkenyl, C₂-C₂₀ substituted alkynyl, C₆-C₁₄ aryl, C₁-C₈ heteroaryl, C₃-C₈ cycloalkyl, C₂-C₈ heterocycyl, C₆-C₁₄ substituted aryl, C₁-C₈ substituted heteroaryl, C₃-C₈ substituted cycloalkyl, and C₂-C₈ substituted heterocycyl. In addition, R can be a carboxyl containing solid support of the formula SS-L-COOH where SS is a solid support and L is a bond or a linker.

Acylation reactions are well known in the art and typically employ the carboxylic acid in activated form such as by reacting the carboxylic acid with an agent such as dicyclohexyl carbodiimide (DCC) in combination with dimethylaminopyridine (DMAP) prior to addition of the alcohol group of the label to facilitate ester formation.

Similarly, if the label contains a carboxyl group, then the alcohol has the formula R-OH where R is as defined above including a solid support and a linker provided that R is not ethylene or acetylene. The reaction proceeds as above with the caveat that the DCC/DMAP is used in combination with the carboxyl group of the label.

Alternatively, acylation of an alcohol group can proceed by converting the carboxyl group to an acid halide such as an acid chloride by conventional means well known in the art. The acid halide is exceptionally reactive and acrylates the hydroxyl group to form the ester. The reaction is typically conducted in an inert solvent in the presence of a base such as diisopropylethylamine (DIEA) to scavenge the acid generated during the reaction.

### Detection Methodology

In one embodiment, detection of a UTI in the patient's urine can be achieved by a hydroxyl-containing solid support covalently linked to a carboxyl-containing label. In the presence of a UTI, the ester bond is cleaved as follows:

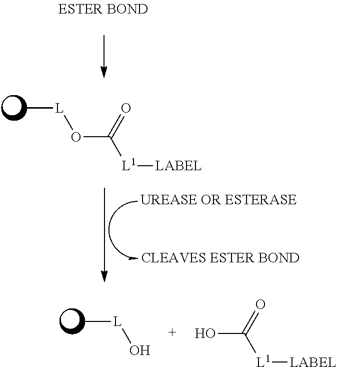

where L and L¹ are independently a bond or a linker group wherein the linker group and LABEL are as defined herein.

In one embodiment, detection of a UTI in the patient's urine can be achieved by a carboxyl-containing solid support covalently linked to a hydroxyl-containing label. In the presence of a UTI, the ester bond is cleaved as follows:

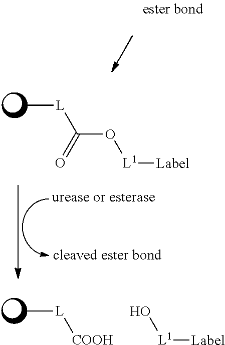

where L, L¹ and label are as defined above.

In both cases, the release of the label from the solid support allows the label to intermix with the urine that is collected in the urine collection bag. Merely assessing for the presence or absence of the label in the urine collection bag rapidly informs the clinician as to whether or not the patient is suffering from a UTI.

Again, in both cases, the solid support can be any suitable support such as microbeads with either hydroxyl or carboxyl pendent groups. Such microbeads are well known in the art and are commercially available.

The above methods provide for facile evaluation of the patient's urinary tract health merely by evaluating the urine of the patient relative to the absence or presence of the label in intact form (no UTI) or in cleaved form (a UTI). So, for the purposes of this invention, a “pro-label” refers to a label comprising an ester bond which remains in intact form regardless of whether that bond to a solid support or not. Likewise, the term “label” refers to a label where the previously bound ester bond has been cleaved. In such cases, it should be noted that pro-labels include a variety of ester bond related compounds.

Further examples of pro-labels include those where the ester bond(s) mask the ability of the label to generate a detectible signal. Cleaving these ester bonds restore the ability of these labels to generate a detectible signal. An example of such pro-labels includes the following:

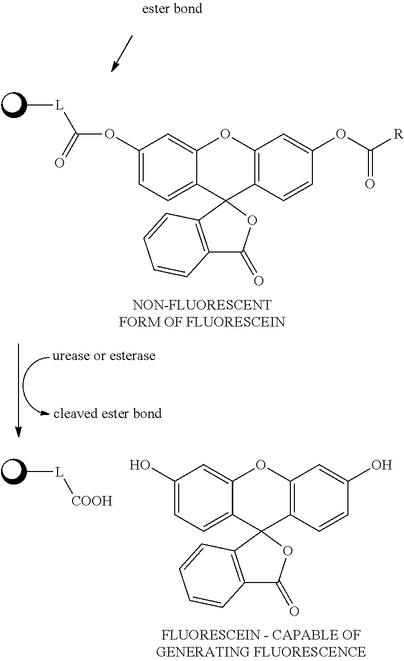

In this embodiment, the detection of a UTI can be measured by evaluating the presence of fluorescence in the urine.

In another embodiment, pro-labels include those where the ester bond links a detectible label to a solid support such that the detectible label is maintained in a first position bound to the support but otherwise is fluorescent. This, in turn, allows the support to be fluorescent. However, in the presence of a UTI, the ester bond is cleaved and the fluorescent label is removed from the solid support and into the urine. This results in the label being placed into a second position (in the urine) whereas the support is no longer fluorescent. An example of such pro-labels includes the following:

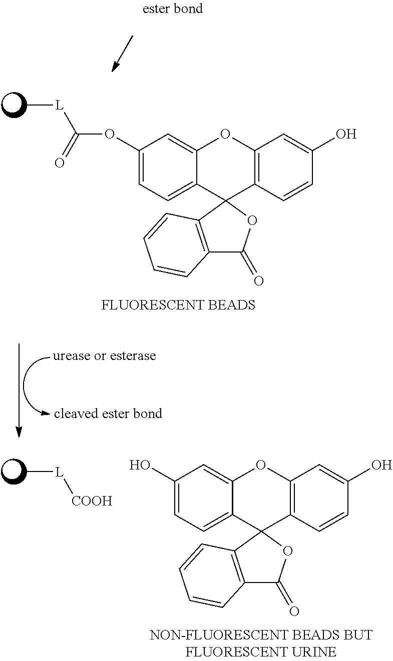

In this embodiment, evaluation of the patient's urine for fluorescence or the beads for lack of fluorescence can be used to determine the presence or absence of a UTI. This is an example where the pro-labels are still capable of generating a detectible signal but such a detectible signal is restricted by covalent binding of the label to a solid support via at least one ester bond.

In another embodiment, pro-labels include those where the ester bond(s) mask the ability of the label to generate a detectible signal and the label is irreversibly bound to a solid support. An example of such pro-labels includes the following:

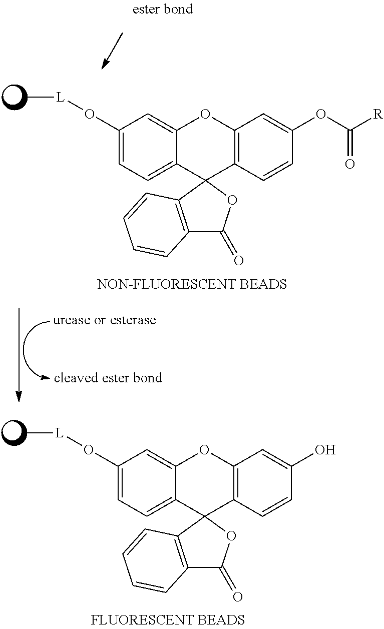

In this embodiment, the pro-label is irreversibly attached to the beads and the label is modified by an ester bond to be a non-fluorescent pro-label. In the presence of urine from a UTI, the beads change from non-fluorescent to fluorescent by conversion of the pro-label to a label simply by cleaving the ester bond. Here, evaluating whether the beads are non-fluorescent or fluorescent will indicate absence or presence of a UTI.

In another embodiment, pro-labels include those where the ester bond(s) mask the ability of the label to generate a detectible signal and the pro-label is maintained either in compound form or as part of a composition containing a suitable carrier. An example of such pro-labels includes the following:

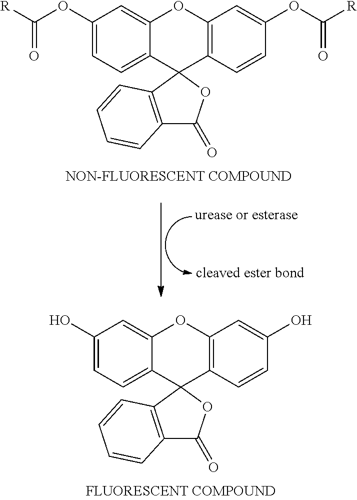

In this embodiment, the pro-label is masked from producing a detectible signal. As such, it can be used directly in a urine collection bag. If the urine is from a patient without a UTI, the pro-label remains intact and generates no signal. If the urine is from a patient with a UTI, then the ester bonds are broken transforming the pro-label into a label capable of generating a detectible signal. In such a case, the urine in the urine collection bag will evidence the fluorescent fingerprint of fluorescein.

As is apparent, there are numerous embodiments in which an ester bond can be coupled to a label to render it as a pro-label and allow for the detection of a UTI in a patient.

### Chambers

In one embodiment, the technology described herein provides for a chamber that allows for consolidation of solid supports into an aqueous permeable space wherein the chamber is defined by a porous flow through under conditions that prevent the solid supports from exiting the chamber. For example, the flow through can be defined by a number of holes or tunnels sized so as to allow passage of fluid but retain the solid supports. An example of such would be a frit or membrane wherein the frit or membrane retains particles of greater than a defined micron size but allows fluid to traverse there through.

In one embodiment, the frits or membranes are placed at or near the proximal or distal end of a connecting device that connects the terminal portion of the catheter to the urine collection bag. Such connectors are well known in the art and are commercially available. For example, suitable connectors are illustrated below (without the solid supports or frits).

As illustrated, the connectors shown above include proximal and distal ribs as well as a lumen passing through the connector. Such connectors are examples of where a proximal and distal frits can be placed preferably near one of the ribs at each end and solid supports containing label bound compounds attached thereto via an ester or carbonate bond are found in a chamber placed between such frits or membranes provided that the loading of the solid supports allow for fluid flow therethrough. The exact configuration and structure of the chamber is not critical provided that it allows for flow through of urine and intimate contact of the urine with solid supports.

Alternatively, in one embodiment, the chamber as defined above is positioned in the catheter and preferably at the distal end of catheter. For example, a porous chamber is integrated or fixed into the catheter lumen so as to ensure that urine passes through the chamber.

In another embodiment, the chamber or the pro-label is placed in the urine collection bag. When placed in a connecting device, the urine contacts the pro-label whether in the chamber or merely added to the bag. If the urine is derived from a patient with a UTI, then the pro-label will be converted to a label that is readily detected by the attending clinician.

### Kits

In one embodiment, the technology described herein provides for a kit that comprises one or more of a catheter, a urine collection bag and/or a chamber as described above; and a pro-label.

In view of the above, the kits described herein can be used in a method for detecting the presence of a urinary tract infection in a patient suspected of having such an infection which method comprises:
a) identifying a patient suspected of having a urinary tract infection;
b) contacting urine from the patient with a solid support that covalently links a fissile label to the support which label breaks in the presence of one or more components in the urine arising from a urinary tract infection;
c) measuring for the presence or absence of label in the urine after said contacting; and
d) based on said presence or absence of label, ascertain that the patient does or does not have a urinary tract infection.

In one embodiment, the kit comprises a solid support comprising a plurality of fissile bonds, each fissile bond is linked to a label, and each fissile bond can be cleaved by a component in urine from a patient suffering a urinary tract infection to release the label into the urine. In one embodiment, the kit comprises a urine collection bag. In one embodiment, the kit comprises a catheter.

In one embodiment, the kit comprises a catheter, a urine collection bag, and a chamber comprising a proximal frit and a distal frit to entrap a solid support within the chamber, wherein the solid support comprises a plurality of fissile bonds, each fissile bond is linked to a label, and each fissile bond can be cleaved by a component in urine from a patient suffering a catheter-associated urinary tract infection.

In one embodiment, the chamber is a connector between the catheter and the urine collection bag. In one embodiment, the chamber is in the urine collection bag. In one embodiment, the label is a fluorescent label, and the fluorescent label has excitation and emission wavelengths of from about 300 nm to about 700 nm. In one embodiment, the pro-label is a fluorescent label, and the fluorescent label has excitation and emission wavelengths of from about 650 nm to about 700 nm. In one embodiment, the label is a pH indicator, an isotope release label, or a solid particle that can affect light scattering. In one embodiment, the fissile bond is an ester bond or a carbonate bond. In one embodiment, the label is a hydroxyl-containing label and the solid support contains a complementary carboxyl group. In one embodiment, the label is a carboxyl-containing label and the solid support contains a complementary hydroxyl group. In one embodiment, the component only found in urine from a patient suffering a catheter-associated urinary tract infection is ammonia, ammonium hydroxide, urease, or a combination of two or more thereof.

### EXAMPLES

### Example 1—In Vitro Test Demonstrating Breakage of Ester Bonds

The following example demonstrates that the generation of ammonia by urease and urea will cleave ester bonds of pro-fluorescent fluorescein compounds to provide for fluorescent molecules.

Two different fluorescein diesters were used in this example. These diesters were fluorescein dibutyrate and fluorescein dioctanoate both of which are commercially available or can be prepared by reacting fluorescein under conventional esterification techniques using as acid anhydrides, acid chlorides, etc. as described herein. The structure of fluorescein diesters is as set forth below:

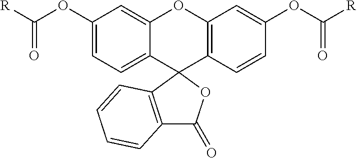

The specific diesters employed are as follows:

A. Fluorescein dibutryate (each R is —CH₂CH₂CH₂CH₃)
B. Fluorescein dioctanoate (each R is —CH₂CH₂CH₂CH₂CH₂CH₃)
The assay was conducted with the following:
   1. Assay Buffer A: 0.5 M Urea in phosphate buffered saline, pH 7 (freshly prepared from solid urea).
   2. Assay Buffer B: Assay Buffer A plus 0.05% bovine serum albumin
   3. Urease: 2000 u/mL in phosphate buffered saline (PBS). This equates to about 40 mg per mL.
   4. Fluorescein diesters are dissolved in water with 1% DMSO.

### Assay Protocol

100 microliters of PBS, Assay Buffer A, or Assay Buffer B were added to wells of a 96 well cluster.
10 microliters of urease were added to the indicated wells.
5 microliters of each diester were added to each well.

The wells were monitored for ten minutes at which time a photograph was taken. The results of that photograph evidence the following:
Buffer A—no fluorescence
Buffer B—minimal fluorescence for fluorescein dibutyrate; no to substantially no fluorescence for fluorescein dioctanoate
Buffer B+urease—fluorescence for both fluorescein dibutyrate and fluorescein dioctanoate.

### Example 2—Second In Vitro Test

The following example demonstrates that normal human urine does not cleave ester bonds of a pro-label but that the same human urine containing the same pro-label plus urease promptly converts the pro-label to a label that generates a distinct detectible signal.

In this example, fluorescein dioctanoate (a non-fluorescent derivative of fluorescein and a pro-label) was combined with normal urine recently obtained from a male volunteer. The urine was divided into two containers. The first container (A) contained fluorescein dioctanoate added as a 1% solution in DMSO. The second container (B) additionally contained urease.

Both samples were maintained at room temperature for approximately 10 minutes and the samples were then evaluated for fluorescence. As is well known, the fluorescence emitted by fluorescein is typically a greenish-yellow color.

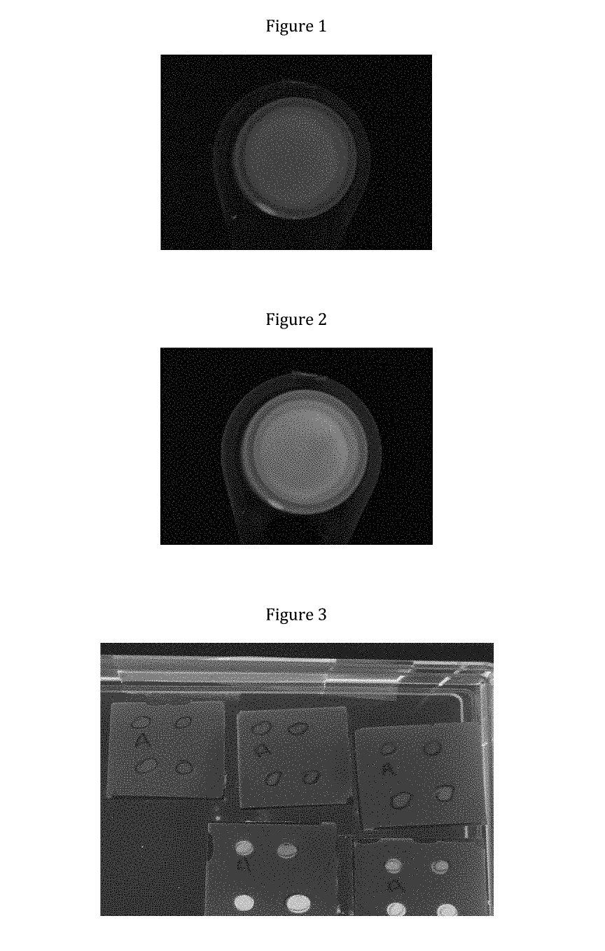

As shown in FIG. 1, container A evidenced no fluorescence having a greenish-yellow color. On the other hand, FIG. 2 shows that container B has a bright greenish-yellow color attributable to fluorescein fluorescence.

The above data demonstrates that urease will react with urea in human urine to generate ammonia that converts the pro-label to a label by breaking the ester bond. Moreover, this conversion provides clear unequivocal evidence that a bacterial infection that generates urease in human urine can be detected by the methods of this invention.

### Example 3—Third In Vitro Test

The following example demonstrates the results of adding P. mirabilis to normal human urine. Specifically, the prior examples demonstrated that ammonia is capable of breaking an ester bond of a profluorescent compound (pro-label) into a fluorescent compound (label). In this example, no exogenous urease was added to the urine. Rather, all of the urease was generated by P. mirabilis. The purpose of this test is to show that a P. mirabilis based UTI will generate sufficient urease and, as a result, sufficient ammonia, to break an ester bond of pro-labels (e.g., in the tests below—fluoresecein diacetate, fluorescein dibutyrate, fluorescein 6,6-dimethylhexanoate, and fluorescein 7,7-dimethylheptanoate) to a label capable of providing a distinct detectible signal (fluorescein).

In this example, P. mirabilis was compared to E. coli, a bacteria that does not produce any urease. In each case, bacteria were incubated in a urine medium over night at approximately 37° C. Five solid supports comprising a silica gel chromatography plates comprising about 250 microns of silica gel over an aluminum backing were spotted with four different fluorescein diesters as noted above. It is contemplated that a glass backing can be used in placed of aluminum in order to direct the UV light from either the bottom or the top of the plate. In each case, the letter “A” was inscribed below the fluorescein diacetate.

Each of these plates were immersed into the following solutions (going from left to right, top row first, bottom row second).

1. A healthy human urine sample with no added bacteria used as a control.
2. A healthy human urine sample with added E. coli bacteria that were not removed prior to contact with the plate.
3. A healthy human urine sample with added E. coli bacteria that were removed from the solution prior to contact with the plate.
4. A healthy human urine sample with added P. mirabilis bacteria that were not removed prior to contact with the plate.
5. A healthy human urine sample with added P. mirabilis bacteria that were removed from the solution prior to contact with the plate.

After incubating the plates in the solutions defined above for a period of time, the plates were removed from the solution and exposed to UV light. The results of this experiment evidence that only those plates containing urine exposed to P. mirabilis regardless of the presence or absence of the bacteria in the solution evidenced significant fluorescence. In contrast thereto, the plates defining the control as well as the E. coli incubated urine evidenced no fluorescence. This substantiates that the P. mirabilis generated sufficient urease that, in turn, generated sufficient ammonia to deacylate each of the profluorescent fluorescein diesters.

### Example 4—Fourth In Vitro Test

Certain E. coli species are a common cause of UTIs. While Example 3 showed that E. coli in a urine solution lacks the ability to cleave ester bonds, in a UTI infection mediated by pathogenic E. coli, the inflammatory sequale of the infection results in leukocyte migration into the urinary tract and subsequent lysis of these invading leukocytes thereby releasing leukocyte esterases. So, unlike Example 3 where E. coli was used in the absence of blood components, this example evaluates the results of whether a blood lysate would deacylate the ester bonds of pro-labels.

Specifically, this example evaluates if these esterases are present in sufficient quantities in the lysate to effect transformation of the pro-labels to labels by deacylation. Accordingly, fluorescein dioctanoate (a non-fluorescent pro-label) was contacted with a blood lysate. A control composition (phosphate buffered saline) containing the same pro-label was prepared.

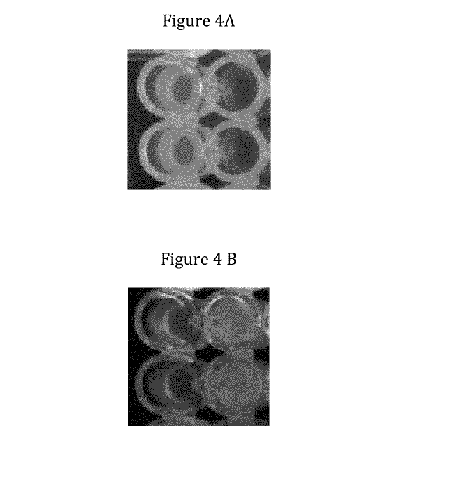

FIG. 4A shows two columns and two rows of test tubes. The first column contains the control and the second column contains the pro-labeled solution. This figure is taken at time equal to zero. As is expected, there is no fluorescence evident in any of the test tubes.

FIG. 4B also shows two columns and two rows. As before, the left column is control and the right column is the blood lysate in the presence of fluorescein dioctanoate. After incubation for 90 minutes and upon the application of UV light, the test tubes containing the fluorescein dioctanoate evidenced fluorescence whereas the control did not. As to the greenish tinge around the control test tubes, it is postulated that this is the result of reflection from the bright green fluorescence of the adjacent tubes (not shown). Those tubes were removed from FIG. 4 so as to allow a direct comparison between the control and fluorescein dioctanoate. Regardless, these results evidence that these pro-labels detect the esterases released during a UTI regardless of the bacterial cause of the UTI.

### Example 5—Preparation of a Solid Support With a Fluorescent Label

Fluorescent label (Compound A) is available from Sigma Aldrich, St. Louis, Mo., USA (e.g., no. 78493)

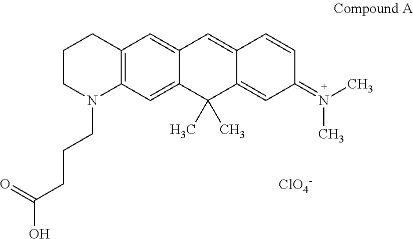

Solid microparticles of poly(2-hydroxyethyl)methacrylate (poly(2-HEMA) or poly(HEMA); molecular weight of about 20,000, or about 300,000, or about 1,000,000, available from Sigma Aldrich, St. Louis, Mo., USA; e.g., product nos. 529265, 192066, and 529257) are combined with fluorescent label (Compound A) under esterification conditions to provide for a labeled microparticle of the formula:

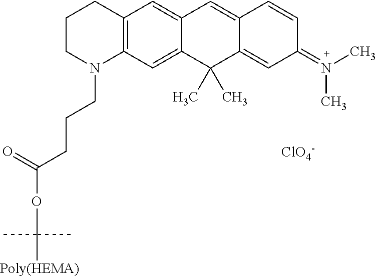

The above labeled microsized polymer is contacted with two different samples of urine. The first sample is normal urine and the second sample is from a patient with a catheter associated urinary tract infection due to Proteus mirabilis. The two samples are separately maintained in contact with the labeled micronized polymer for a sufficient period of time to permit breaking of the ester bonds by any ammonia/ammonium hydroxide in the sample produced by Proteus mirabilis. The two samples are removed from the microsized polymer and checked for fluorescence arising from the presence of Compound A in the urine. It is expected that the urine from the patient with the UTI will fluoresce due to the presence of Compound A (construed to be a positive test result that the patient has a UTI due to Proteus mirabilis) whereas the normal urine will not.

What is claimed is:

1. A composition comprising urine and a solid support comprising a plurality of fissile ester bonds, each fissile bond is linked to a label so as to provide for a pro-label, and each fissile bond is stable in urine from a patient not suffering from a urinary tract infection but is cleaved by a component in urine from a patient suffering a urinary tract infection to convert the pro-label to a label capable of generating a distinct detectible label in said urine.

2. The composition of claim 1, wherein the label is a fluorescent label.

3. The composition of claim 1, wherein the label is a pH indicator, an isotope release label, or a solid particle that can affect light scattering.

4. The composition of claims 1, wherein the solid support is retained in a chamber that allows for the passage of urine there through.

5. A method for detecting a urinary tract infection (UTI) in a patient suspected of being afflicted with a UTI which method comprises:
contacting urine of said patient with a pro-label having one or more ester functionalities wherein said ester functionalities remain intact in the presence of normal urine but are cleaved in the presence of urine associated with a UTI to provide a label that is capable of providing for a distinct detectible signal evidencing the presence of a UTI;
maintaining said pro-label in contact with said urine under conditions wherein the ester bond or bonds is/are cleaved in the presence of a UTI;
assessing for the presence or absence of said signal; and
correlating the presence or absence of said signal to the presence or absence of a UTI in said patient.

6. A method for detecting a urinary tract infection (UTI) in a patient suspected of being afflicted with a UTI which method comprises:
contacting urine of said patient with a pro-fluorescent label having one or more ester functionalities wherein said ester functionalities remain intact in the presence of normal urine but are cleaved in the presence of urine associated with a UTI to provide a label that is capable of generating a distinct detectible fluorescent signal evidencing the presence of a UTI;
maintaining said pro-fluorescent label in contact with said urine under conditions wherein the ester bond or bonds is/are cleaved in the presence of a UTI;
assessing for the presence or absence of said fluorescent signal; and
correlating the presence or absence of said signal to the presence or absence of a UTI in said patient.

7. A method for detecting a urinary tract infection (UTI) in a patient suspected of being afflicted with a UTI which method comprises:
contacting urine of said patient with a pro-fluorescent fluorescein label having one or more ester functionalities wherein said ester functionalities remain intact in the presence of normal urine and are cleaved in the presence of urine associated with a UTI to provide a fluorescent fluorescein label capable of providing a distinct detectible signal evidencing the presence of a UTI;
maintaining said pro-fluorescent fluorescein label in contact with said urine under conditions wherein the ester bond or bonds is/are cleaved in the presence of a UTI;
assessing for the presence or absence of said signal; and
correlating the presence or absence of said signal to the presence or absence of a UTI in said patient.

8. A method for detecting the presence of a urinary tract infection in a patient suspected of having such an infection which method comprises:
a) identifying a patient suspected of having a urinary tract infection;
b) contacting urine from the patient with a solid support that covalently links a label to the solid support which linkage is cleavable in the presence of a urinary tract infection;
c) measuring for the presence or absence of label in the urine after said contacting; and
d) ascertaining that the patient does or does not have a urinary tract infection based on said presence or absence of label.

9. A method to treat a patient suspected of suffering from a urinary tract infection, the method comprising
a) contacting urine from the patient with a solid support that covalently links a label to the support which linkage is cleavable in the presence of a urinary tract infection;
b) measuring for the presence or absence of label in the urine after said contacting;
c) ascertaining that the patient does have a urinary tract infection based on presence of label in the urine; and
d) administering medication to the patient to diminish or eradicate the urinary tract infection.

10. The method of claim 9, further comprising confirming the presence or absence of label in the urine in a laboratory assay after step c) but prior to step d).

11. A method for detecting the presence of a urinary tract infection (UTI) in a patient suspected of having such an infection which method comprises:
a) identifying a patient suspected of having a UTI;
b) providing a solid support having a label covalently linked thereto which linkage is cleavable in the presence of a UTI, wherein said label emits a detectable signal and said solid support is in a first position;
c) contacting said solid support with urine from said patient such that, in the presence of a UTI, the linkage is cleaved and the label moves to a second position;
d) measuring for the presence or absence of the signal in the first position and/or the second position after said contacting; and
d) ascertaining that the patient does or does not have a urinary tract infection based on said presence or absence of label in the first position and/or the second position.
